(19) United States
(12) Patent Application Publication
(10) Pub. No.: US 2004/0015202 A1
(43) Pub. Date: Jan. 22, 2004

(54) Combination epidural infusion/stimulation method and system

(76) Inventors: Gilbert S. Chandler III, Gainesville, GA (US); L. Todd Stewart, Gainesville, GA (US)

Correspondence Address: Jack D. Todd, Morris, Manning & Martin, LLP, 3343 Peachtree Road, N.E., Atlanta, GA, 30326-1044, US

(21) Appl. No.: 10/461,924

(22) Filed: Jun. 13, 2003

Related U.S. Application Data
(60) Provisional application No. 60/388,963, filed on Jun. 14, 2002.

(51) Int. Cl. A61N 1/18

(52) U.S. Cl. 607/46; 607/3

(57) ABSTRACT

A system for providing pain relief to a person includes a spinal cord stimulation (SCS) generator, an infusion pump, a controller for selectively controlling generation of an electrical stimulation signal from the SCS generator and for selectively pumping medication from the infusion pump, and a lead adapted for placement along the spinal cord of the person. The lead includes electrodes connected to the SCS generator for providing SCS to a first location on the spinal cord of the person and an infusion port for delivering medication from the infusion pump to a second location on the spinal cord. A corresponding method of providing pain relief includes implanting a combination infusion/spinal cord stimulation device along the spinal cord of the person and then, simultaneously, alternatively, or serially, infusing fluid medication at a first location of the spinal cord using the device and providing SCS at a second location using the same device.

### CROSS REFERENCE TO RELATED APPLICATION

This application claims priority under 35 U.S.C. 119 to the benefit of the filing date of Chandler et al, U.S. patent application Ser. No. 60/388,963, which was filed on Jun. 14, 2002, titled “COMBINATION EPIDURAL INFUSION/STIMULATION DEVICE,” which is incorporated herein by reference.

### BACKGROUND OF THE INVENTION

1. Field of the Invention

The present invention relates generally to pain treatment and, more specifically, to a combination treatment process or apparatus using both spinal cord (epidural or intrathecal) infusion and spinal cord stimulation simultaneously, alternately, or serially.

2. Description of the Prior Art

Mankind has battled pain with a variety of devices and techniques throughout recorded history. As populations continue to grow, the prevalence of both acute and chronic pain requiring medical attention continues to escalate as well. These conditions exact a heavy toll on the individual sufferers, their family and friends, their community, and society as a whole. Pain impairs and disables in both a short or long term time period via psychological, physical, behavioral, vocational, social, and economic avenues, or combinations thereof. By some estimates, fifteen (15) to twenty (20) percent of the population suffers from acute pain and twenty-five (25) to thirty (30) percent of the population suffers from chronic pain. See, e.g., The Management of Pain, by John J. Bonica, Vol. I, 2^{nd} Edition, p. 2 (1990).

Various pain treatment devices and techniques have been documented throughout recorded history. Some ancient forms of treatment devices and techniques are still employed today, including counter-stimulation, acupuncture, herbs, opioids, heat, and massage. Advances in anatomy and physiology have allowed a greater understanding of pain pathways. Multiple classes of medications have been developed to target different portions of these pathways to alter the sensations and/or responses to painful stimuli. These drugs are delivered by different routes including oral, rectal, intra-muscular, trans-mucosal (oral or nasal), transdermal, intravenous, epidural, intrathecal, or by inhalation. Modern imaging techniques are often used to demonstrate pathology non-invasively. The invention of the hollow needle and, more recently, advances in microelectronic technology have further added to the diagnostic and therapeutic acumen available in the battle against pain and its devastating consequences.

Physical pain is generally divided into two broad categorizes: nociceptive pain and neuropathic pain. Nociceptive pain is generated by pathophysiological processes via specialized sensory nerve endings or receptors. Stated more simply, the body is covered in millions of free nerve endings (or “nociceptors”), whose specific function is to relay pain signals to the spinal cord. These nociceptors convert painful stimulation (e.g., incisions, pressure, burns, fractures, sprains, etc.) into neuro-electric “current,” which travels via specific pathways (e.g., A-delta or C-fibers) to the spinal cord. These fibers end in the spinal cord, where this signal is processed. Specific receptors in the spinal cord then transmit the pain signal to the brain. This entire process is basically an electrochemical relay race in which the pain “baton” is exchanged between the fibers and the spinal cord at an area called the “dorsal horn.” This complex process is affected by the stimulus intensity, duration, and localized tissue chemicals at the site of the injury. In addition, certain messengers (e.g., glutamate, aspartate, CGRP, CCK, and Substance P) act to “boost” the pain signal. Conversely, pain reduction techniques seek to interfere with this electro-chemical relay by blocking pain receptors or by chemically “tying-up” the particular messengers. Chemicals used to tie up such messengers include serotonin, opiods, norepinephrine, local anesthetics, and gamma-aminobutryic acid (GABA).

In contrast, neuropathic pain is produced by afferent fibers directly secondary to damage by physical, chemical or physiologic processes. Examples of neuropathic pain include post herpetic neuralgia, reflex sympathetic dystrophy/causalgia (nerve trauma), and entrapment neuropathy (carpal tunnel syndrome).

A first method for treating severe pain is the continuous administration of medication, such as a mixture of narcotics and local anesthetics, delivered epidurally or intrathecally. An epidural administration of medication typically involves placing a catheter in the epidural space (i.e., the space just outside of the thecal sac that contains the spinal cord) and delivering medication by intermittent bolus or continuous infusion. The administration of medication reduces transmission of the pain signal(s) via receptors in the spinal cord or by local anesthetic action at the nerve root level. Intrathecal therapy is similar to epidural treatment; however, it involves the delivery of medication directly within the thecal sac. The thecal sac is a balloon that covers the brain and spinal cord and is covered with cerebrospinal fluid. When medication is delivered directly into this fluid-filled sac, the medication goes directly to the receptors where it is needed to work. A benefit of intrathecal delivery (in contrast with epidural, oral, or transdermal delivery) is the fact that less dosage is required to have the desired pain-reducing effect. This, in turn, leads to a reduction of side effects associated with the taking of medication. For example, one milligram of intrathecal-delivered morphine has the same effect as 150 to 300 milligrams given orally.

Epidural and intrathecal therapy provide neuraxial modes of drug delivery, which have been demonstrated to treat pain effectively from multiple etiologies, including acute perioperative (i.e., before, during, and after operation) pain and chronic neuropathic pain. For example, epidural analgesia has been shown to improve patient satisfaction, decrease pain scale scores, lower morbidity from multiple organ systems, decrease venous thrombosis, and allow for earlier discharge when employed for perioperative pain control. The use of epidural analgesia is largely limited by costs, intensive maintenance requirements, and intolerable side effects (e.g., Pruritis, nausea/vomiting, sedation, urinary retention, respiratory depression, and weakness) as well as infectious risks (e.g., meningitis) and the remote possibility of bleeding around the spinal cord, potentially leading to paralysis.

Long term continuous infusion frequently increases the patient's tolerance to the medication, which requires an ever-increasing medication dose to maintain effectiveness. With increased medication dosage: 1) side effects tend to become more intense—limiting the increase in dosage; 2) intervention via refill of the drug or maintenance of the drug pump becomes more frequent—increasing cost, infectious risks, and patient discomfort—and decreasing convenience and satisfaction for the patient; and 3) effectiveness tends to degrade despite maximizing delivery of the drug concentration or quantity.

A second common method of treating severe pain is a technique known as dorsal column stimulation or spinal cord stimulation (hereinafter “SCS”). This second method of pain treatment is used primarily for the treatment of pain of neuropathic origin from a variety of pathologic states. There are many clinical studies that support the use of SCS for the treatment of back and extremity pain of neuropathic origin; thus, use of SCS has typically been limited to the treatment of severe chronic (long-term) pain symptoms, as is common with prior back surgery followed by persistent back or leg pain. Such studies provide documented improvement in patient function coupled with decreased pain scores and decreased medication consumption. The precise SCS mechanism of action was once thought to be simply based upon the inhibition of noxious transmission via electrical stimulation of the dorsal columns of the spinal cord based upon the gate control theory of Melzack and Wall. See Textbook of Pain, 3^{rd} Edition, edited by Melzack and Wall, p. 279 (1994).

The SCS pain relief mechanism now is believed to be multi-factoral involving various peptides, neurotransmitters, sympathetic modulation, and/or action on descending analgesia pathways in addition to its action on the pain “gate,” as originally believed. Generally the SCS treatment side effects are less than that associated with an epidural catheter infusion of medications since SCS is a non-drug (i.e., non-medication) treatment procedure. On the other hand, however, accurate placement of the SCS electrodes can be more difficult than placement of an epidural catheter and, on rare instances, patients dislike the sensation caused by the SCS stimulation.

Although, recently, SCS has been used with some success in the non-surgical treatment of peripheral vascular disease (PVD) and anginal (cardiac) pain, SCS has historically not been considered for use in conjunction with postoperative pain because SCS is not thought to provide relief for nociceptive pain. Based on limited studies, the inventors of the present invention have determined that SCS appears to be effective in the treatment of some types of nociceptive pain, such as pain following total knee replacement. Based on these limited studies, SCS treatment seems to provide adequate analgesia, virtually no side effects, low maintenance requirements, patient control of analgesia (e.g., by allowing the patient to adjust the level of stimulation intensity on demand), and good patient satisfaction, all of which allowed for rapid rehabilitation postoperatively.

A disadvantage of long term use of SCS treatment, however, is the fact that such use is associated with tachyphylaxis (i.e., the rapid appearance of progressive decrease in response following repetitive administration of a pharmacologically or physiologically active substance), because the stimulation delivered becomes less effective in reducing pain with time over the term of months or years of continued treatment. The continuous use of SCS treatment makes tachyphylaxis more likely to occur and generally hastens the onset of tachyphylaxis. Further, continuous and more intense SCS treatment shortens the battery life of the stimulation generator, which necessitates more frequent intervention (i.e., change or recharge of battery) and which, correspondingly, increases treatment costs, infectious risks, and patient discomfort.

While the separate use of SCS and epidural (or intrathecal) treatments and devices are suitable for various pain treatments, it would be advantageous to be able to utilize both of the treatments simultaneously, alternately, or serially, as desired, to provide effective relief of pain symptoms while minimizing the side effects and disadvantages associated with the exclusive use of either such treatment, as described briefly above.

### SUMMARY OF THE INVENTION

The present invention provides a new pain treatment process, apparatus, and system that allows the combined use of SCS and epidural (or intrathecal) infusion simultaneously, alternately, or serially, as desired. A combination spinal cord (epidural or intrathecal) infusion/stimulation device provides for stimulation of the spinal cord and a medication infusion (epidural or intrathecal) at sites in close proximity to one another. Such a device is designed to be located along the spinal cord with the epidural/intrathecal infusion site typically above or below the proximate stimulation site. Typically, the sites for SCS and infusion are not exactly the same since each treatment has a specific, and usually different, “sweet spot” for addressing and alleviating pain originating from any particular location of the body. Preferably, such device is placed in the desired spinal location in a single procedure and then operated to provide either or both pain treatments, as desired.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways and is only limited by the claims attached hereto. Also, it is to be understood that the phraseology and terminology employed herein are for the purpose of the description and should not be regarded as limiting the scope of the present invention.

In a first aspect of the present invention, a system for providing pain relief to a person comprises a spinal cord stimulation (SCS) generator adapted to provide an electrical stimulation signal, an infusion pump adapted to pump fluid medication, a controller for selectively controlling generation of the electrical stimulation from the SCS generator and for selectively controlling pumping of the medication with the infusion pump, and a lead adapted for placement along the spinal cord of the person, the lead including at least one electrode in electrical communication with the SCS generator for providing SCS to a first location on the spinal cord and an infusion port in fluid communication with the infusion pump for delivery of the medication to a second location on the spinal cord.

In a feature of this aspect of the invention, the SCS generator, the infusion pump, and the controller are contained within a common housing. In another feature, the SCS generator, the infusion pump, and the controller are adapted for implanting within the body cavity of the person. In an alternative feature, the SCS generator, the infusion pump, and the controller are adapted for remaining outside the body cavity of the person.

In yet another feature of the first aspect of the invention, the lead includes a plurality of electrodes. A preferred arrangement of such electrodes is linear; however, many other arrangements are suitable and may be chosen, as desired. Preferably, each of the plurality of electrodes is connected to the SCS generator using a conductive wire. In a further feature of this first aspect of the invention, the infusion port is in fluid communication with the infusion pump using a lumen.

In yet a further feature of this aspect of the invention, the SCS generator and the infusion pump are contained within a common housing, a plurality of electrodes are connected to the SCS generator using wires, the infusion port is in fluid communication with the infusion pump using a lumen, and the wires and the lumen are contained within a tubular element extending from the common housing to the lead.

In one preferable arrangement for treating pain originating from one part of the body, the first location is at approximately segment T10 of the spinal cord and the second location is at approximately segments L1 or L2 of the spinal cord. Alternatively for treating pain originating from another part of the body, the first location is at approximately segment C3 or C4 of the spinal cord and the second location is at approximately segments C6 or C7 of the spinal cord. In a feature of the invention, the second location is within the epidural space or the intrathecal sac of the spinal cord.

In another feature of the first aspect of the invention, the electrodes are distally located relative to the infusion port. In an alternative embodiment, the electrodes are proximally located relative to the infusion port.

In yet a further feature, the lead further comprises a steering lumen. In another feature, the electrodes and the infusion port are within a paddle-shaped element adapted for placement along the spinal cord via open laminotomy or via a modified needle.

In yet a further feature of the first aspect, the system further comprises an electrode connector housing having at least one electrode connector lead and an SCS adapter in electrical communication with the SCS generator, the SCS adapter having an electrode connector coupling adapted for engaging the at least one electrode when the electrode connector housing is engaged with the SCS adapter.

In a second aspect of the present invention, an apparatus adapted for placement along the spinal cord of the person for use in providing pain relief to the person, in combination with a spinal cord stimulation (SCS) generator adapted to provide an electrical stimulation signal and an infusion pump adapted to pump fluid medication, the SCS generator and the infusion pump each controlled by a controller for selectively controlling generation of the electrical stimulation from the SCS generator and for selectively controlling pumping of medication by the infusion pump, comprises a tubular housing, a plurality of electrodes on an outer surface of the tubular housing, the electrodes in electrical communication with the SCS generator for providing SCS to a first location on the spinal cord, and an infusion port extending through the tubular housing, the infusion port in fluid communication with the infusion pump for delivery of medication to a second location on the spinal cord.

In a feature of the second aspect, each of the plurality of electrodes is connected to the SCS generator using a wire. Further, the infusion port is in fluid communication with the infusion pump using a lumen.

In another feature, the electrodes are distally located relative to the infusion port. Alternatively, the electrodes are proximally located relative to the infusion port.

In yet a further feature, the apparatus further comprises a steering lumen.

Additionally, in a feature of this aspect of the invention, the electrodes and the infusion port are within a paddle-shaped element adapted for placement along the spinal cord via open laminotomy or via a modified needle.

A third aspect of the present invention discloses a method of providing pain relief to a person, comprising the steps of implanting a combination infusion/spinal cord stimulation device along the spinal cord of the person, providing fluid medication at a first location of the spinal cord using the device, and providing electrical stimulation at a second location of the spinal cord using the device.

In a feature of the invention, the steps of providing fluid medication and providing electrical stimulation occur simultaneously. In other features, the steps of providing fluid medication and providing electrical stimulation are repeated alternatively or serially.

In yet a further feature of this third aspect of the invention, the first location is either within the epidural space or the intrathecal sac of the spinal cord.

To accomplish the above and related functions, the invention may be embodied in the form illustrated in the accompanying drawings, attention being called to the fact, however, that the drawings are illustrative only, and that changes may be made in the specific construction illustrated without departing from the scope of the present invention.

### BRIEF DESCRIPTION OF THE DRAWINGS

Various other features and attendant advantages of the present invention will become fully appreciated as the same becomes better understood when considered in conjunction with the accompanying drawings, which are not necessarily drawn to scale but in which like reference numbers designate the identical or similar parts throughout the several views, and wherein:

FIG. 1 illustrates a plan view of the present invention located in the spinal cord of a person;

FIG. 2 illustrates a diagrammatic detailed side view of the arrangement of a spinal cord of a person;

FIG. 3 illustrates an enlarged view of the body of one embodiment of the device of the present invention;

FIG. 4 illustrates an enlarged view of the body of a second embodiment of the device of the present invention;

FIG. 5 illustrates an enlarged view of the body of a third embodiment of the device of the present invention;

FIG. 6 illustrates an enlarged view of the body of a fourth embodiment of the device of the present invention; and

FIG. 7 illustrates a preferred external connector element for use with one of the devices of the present invention.

### DETAILED DESCRIPTION OF THE INVENTION

The present invention will now be described more fully hereinafter with reference to the accompanying drawings, in which preferred embodiments of the invention are shown. This invention, however, may be embodied in many different forms and should not be construed as limited to the embodiments set forth herein; rather, these embodiments are provided so that this disclosure will be thorough and complete, and will fully convey the scope of the invention to those skilled in the art.

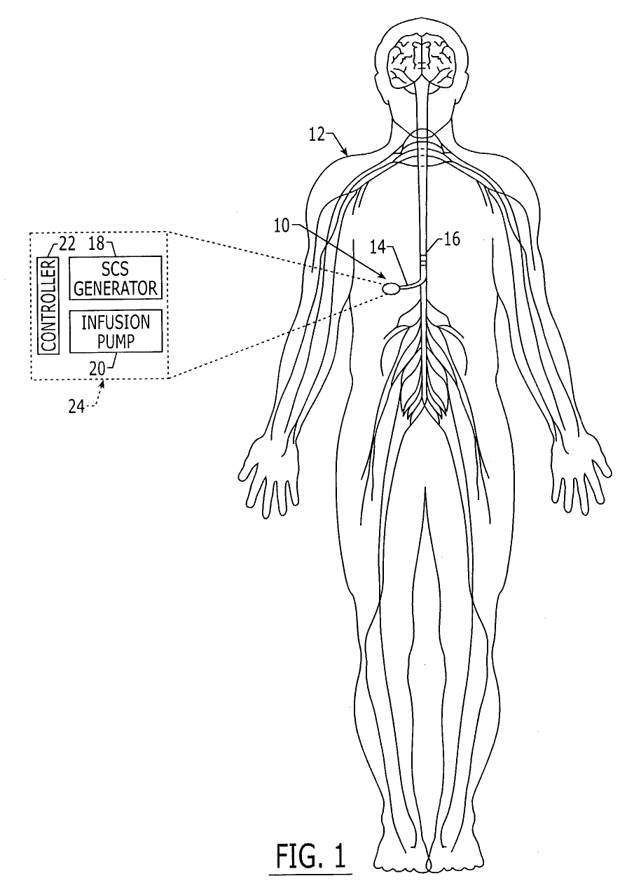

In accordance with a preferred embodiment, FIG. 1 illustrates a combination epidural infusion/spinal cord stimulation device 10 of the present invention. The device 10 is illustrated diagrammatically implanted in a person 12 with a combined infusion and stimulation lead 14 inserted in or along the epidural space of the spinal cord 16 of the person 12. As shown in the blow-up section of FIG. 1, the device 10 includes an SCS generator 18 and an infusion pump 20. In one embodiment, the SCS generator 18 and the infusion pump 20 are separate units, as illustrated, and each has its own controller (not shown). In an alternate embodiment, the SCS stimulation generator 18 and the infusion pump 20 are controlled by a central controller 22, all of which is housed within a combined or single housing 24. In one embodiment, the SCS generator 18 and the infusion pump 20 are implanted within the body cavity of the person 12. In another embodiment (not shown), the SCS generator 18 and the infusion pump 20 are maintained outside the body cavity of the person 12, in which case lead lines from the SCS generator 18 and the infusion pump 20 run to and are coupled with the lead 14, which is entirely or at least substantially implanted within the person 12. Although not preferred, it is possible to implant one of the SCS generator 18 and the infusion pump 20 and have the other unit external to the body cavity of the person 12. It should be understood that the SCS generator 18 and the infusion pump 20 may be of conventional design, such as those manufactured by Medtronic, Inc. of Minneapolis, Minn. Techniques for implanting the device 10, such as percutaneous placement via a needle or modified needle or via an open surgical process, will be known to those skilled in the art and are considered conventional.

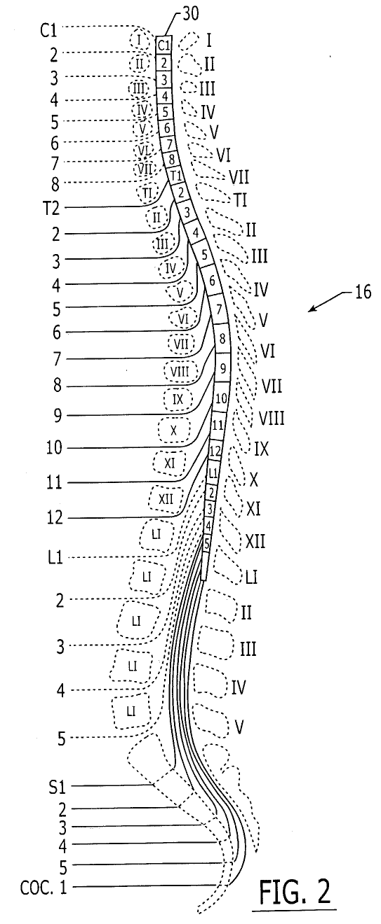

Turning now to FIG. 2, a side view of a typical spinal cord 16 of a person is illustrated. The vertebral column segments 30 of the spinal cord 16 are labeled in conventional manner for reference. The “Cervical” section of the spinal cord includes segments C1 to C8 (C standing for Cervical and numbering proceeding in order from the head down). The Cervical spine is the origin of nerves that go to the arms. The “Thoracic” section of the spinal cord includes segments T1 to T12. The “Lumbar” section of the spinal cord includes segments L1 to L5 and the “Sacrum” includes segments S1 to S5.

It has been found that the ideal locations for placing the infusion catheter for the pump 20 and the stimulation lead for the generator 18 in relation to the spinal cord 16 are generally near to or proximate each other, but usually not in the exact same location along the spinal cord 16. It has also been determined that the ideal locations for placing the catheter and stimulation lead varies significantly depending upon what area of the body is experiencing pain. For example, for treating knee pain, SCS treatment is generally most effective when placed at approximately the T10 segment of the spinal cord 16 while epidural infusion is generally most effective when applied at segment L1 or L2 in the spinal cord 16. In another example, for treating pain in the upper extremities, SCS treatment is generally most effective when placed at approximately the C3 or C4 segment of the spinal cord 16 while epidural infusion is generally most effective when applied at segment C6 or C7 in the spinal cord 16.

A preferred embodiment of a combination proximal port infusion/stimulation apparatus 40 is illustrated in FIG. 3. In this embodiment of the invention, a localized region of pain (e.g., knee, hip or upper extremity pain) is treated simultaneously, serially, or alternately with infusion and SCS provided by the combination proximal port infusion/stimulation apparatus 40. The apparatus 40 comprises a generally tubular element 42, such as formed from silicon or plastic. The apparatus 40 includes a small central steering lumen 44 for use with a lead blank/steering wire (not illustrated) in a conventional manner. The apparatus 40 further comprises an internal infusion lumen 46 for delivering infusion medication. The infusion lumen is connected to an infusion pump 20 (as shown in FIG. 1) and ends at an infusion opening or port 48. The apparatus 40 also advantageously includes a plurality of electrodes 50a, 50b, 50c, 50d, each of which is connected or coupled to the generator 18 (from FIG. 1) by a plurality of wires or leads 52, for the SCS treatment modality. Preferably, four electrodes 50 in a line are utilized with the apparatus 40. The spacing or distance d between the port 48 and the middle of the plurality of electrodes 50 is determined based on the type of pain being experienced by the patient and the locations of the relevant infusion and SCS sites for treating such pain. Such distance d is customizable for a particular person, or standard distances between a typical infusion treatment location on an average spinal cord and the SCS treatment location on an average spinal cord for a particular type of pain may be used. For example, an apparatus 40 designed for use in treating knee pain has a distance d roughly equal to the distance between segment T10 (SCS treatment location) and segments L1 or L2 (infusion location) of the spinal cord 16. In another example, an apparatus 40 designed for use in treating pain in the upper extremities has a distance d roughly equal to the distance between segments C3 or C4 (SCS treatment location) and segments C6 or C7 (infusion location) along the spinal cord 16.

In operation, the apparatus 40 must first be inserted into the body of the person receiving pain treatment in conventional manner. The apparatus 40 is located in the desired spinal location so that the infusion port 48 is adjacent the desired spinal cord segment for receipt of infusion medication (either epidurally or intrathecally) and so that the electrodes 50 are adjacent the desired spinal cord segments for receipt of SCS treatment. The infusion treatment medication(s) are pumped through the lumen 46 and infused through the port 48 by operation of the pump 20, as desired. Electrical stimulation is provided by the SCS generator sending an electrical signal through lead wires 52 to the electrodes 50. As stated previously, infusion treatment and SCS treatment are controlled by their respective controllers or by a combined controller 20, as shown in FIG. 1. The sequence and timing of infusion treatment and SCS treatment in any given case is varied and controllable by the consulting physician and, in some situations, by self-regulation by the patient. Advantageously, infusion treatment and SCS treatment is applied simultaneously, alternatively, or serially, as desired by the treating physician. By alternating such treatments, it is believed that the onset of tachyphylaxis for both infusion medication and SCS treatment is delayed. Further, it is believed that the simultaneous application of both infusion medication and SCS treatment enables lower dosage and electrical stimulation requirements to achieve the same pain reductions.

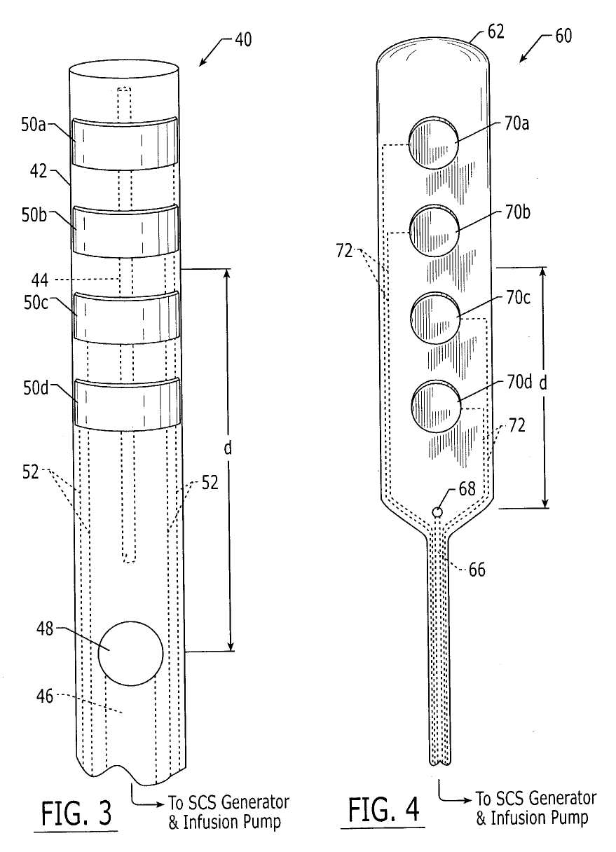

A second embodiment of a combination proximal port infusion/stimulation apparatus 60 is illustrated in FIG. 4. In this embodiment, the apparatus 60 comprises a paddle-shaped element 62, which is placed via open laminotomy or via a modified needle (not illustrated) in the desired spinal location. Like apparatus 40, apparatus 60 includes an infusion lumen or tube 66 and an infusion port 68 connected or coupled to the pump 20 (from FIG. 1). Likewise, the apparatus 60 includes a plurality of electrodes 70a, 70b, 70c, 70d connected or coupled to the generator 18 by a plurality of wires or leads 72. Again, preferably, four electrodes 70 are utilized with the apparatus 60; however, fewer or more electrodes may also be used and still fall within the scope of the invention. Once implanted, the apparatus 60 operates and is operated and controlled in a manner similar to that of apparatus 40.

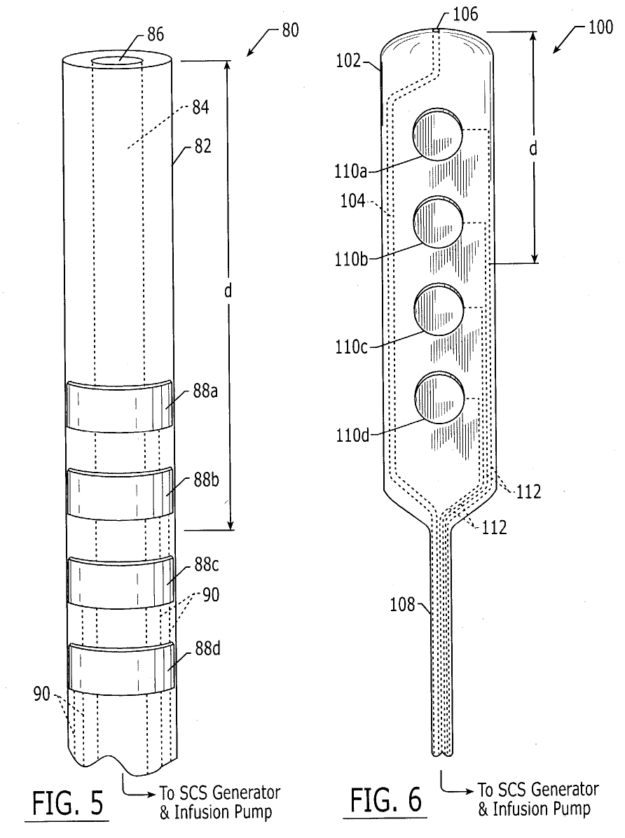

A first embodiment of a combination distal port infusion/stimulation apparatus 80 is illustrated in FIG. 5. The apparatus 80 is functionally the same as the apparatus 40, but with the infusion port and SCS electrodes sites reversed. The apparatus 80 includes a tubular element 82 having an internal infusion lumen 84 with an infusion port 86. The tubular element 82 includes a plurality of electrodes 88a, 88b, 88c, 88d, again coupled to the generator 18 (from FIG. 1) by a plurality of wires or leads 90.

A second embodiment of a combination distal port infusion/stimulation apparatus 100 is illustrated in FIG. 6. In this embodiment the apparatus 100 is a paddle shaped element 102, similar to element 62, but with the infusion port and SCS electrodes sites reversed. The apparatus 100 includes an infusion lumen 104, having an infusion port 106 at one end and coupled to the infusion pump 20 (as shown in FIG. 1) at the other end via a tubing element 108. The element 102 includes a plurality of SCS electrodes 110 and a plurality of wires or leads 112 connecting or coupling the electrodes 110a, 110b, 110c, 110d to the SCS generator 18 (from FIG. 1) through the element 108.

With regard to each of the apparatuses 40, 60, 80, 100 described in FIGS. 3-6, it is preferred that four electrodes in a linear arrangement be used. However, it should be understood by those skilled in the art that fewer or more electrodes and various arrangements (other than linear) of such electrodes may also be used to advantage and still fall within the scope of the present invention.

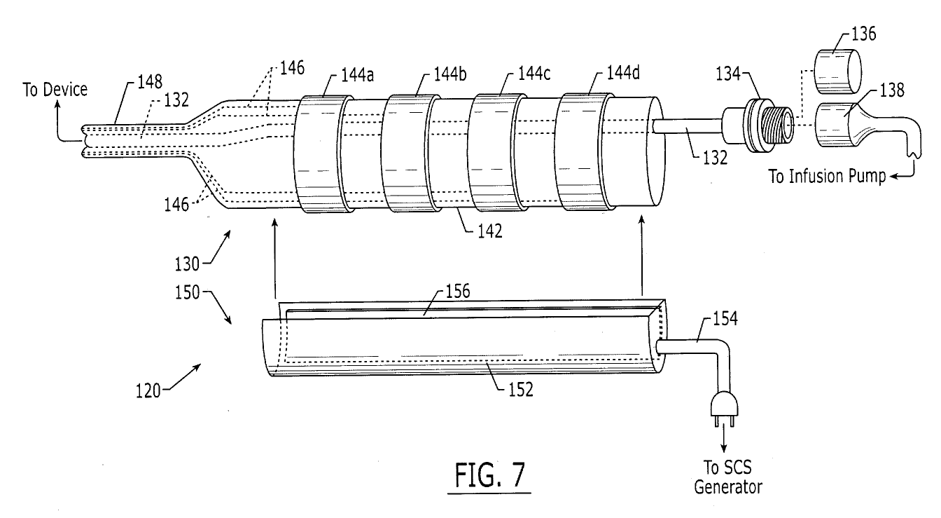

FIG. 7 illustrates a preferred external connector element 120 for use with the combination infusion/stimulation devices of the present invention. The external connector element 120 includes a primary component 130 and an SCS adapter 150. The primary component 130 includes a lumen 132 and a male pump connector 134. The male pump connector 134 is capped with cap 136 when not in use and, when in use, adapted to mate with a female pump connector 138 that connects with a medication infusion bag (not shown), which is controlled by infusion pump 20 (from FIG. 1). At the other end of the primary component 130, the lumen 132 extends through an electrode connector housing 142 that has four external electrode connector leads 144a, 144b, 144c, 144d. Preferably, each external electrode connector lead 144 comprises a plate-like surface that extends around the outer circumference of the. housing 142. Each electrode connector lead 144 is also electrically connected to a respective wire or lead 146 that extends within the electrode connector housing 142 and on through a more narrow tubular element 148, that eventually intersects with the tubular element of the combination infusion/stimulation device of the present invention. Each respective wire or lead 146 eventually connects with an electrode of the combination infusion/stimulation device. Likewise, the lumen 132 continues through the tubular element 148 to the combination infusion/stimulation device.

The SCS adapter 150 includes a housing 152 and a plug connector 154. The plug connector 154 is designed to connect electrically with the SCS generator 18 (from FIG. 1). The housing 152 includes an electrode connector coupling 156, which is a plate-like electrical surface that extends around the inner circumference of the housing 152 and which is electrically connected with the plug connector 154. The housing 152 is adapted to fit around or about housing 142 in such a manner that electrode connector coupling 156 makes electrical contact with all four external electrode connector leads 144a, 144b, 144c, 144d, when twisted or snapped into engagement with the housing 142. Thus, it is preferred that the electrode connector coupling 156 be as long as the four external electrode connector leads 144. Each end of the housing 152 is further adapted to allow the lumen to extend therethrough without interference when the two housings 142, 152 are interconnected.

Although not specifically illustrated above, a combination intrathecal infusion/spinal cord stimulation apparatus is also contemplated within the scope of the present invention. Since it is preferred that SCS not be provided in the thecal sac of the spinal cord, such an apparatus requires an additional lead or lumen with an infusion port for extension into the hecal sac of the spinal cord. Such lead or lumen extends from the infusion port of the apparatus or, if the apparatus has no infusion port, extends separately away from the electrode section of the apparatus for insertion into the thecal sac of the spinal cord. With such a device, it is possible to deliver medication intrathecally while at the same time providing SCS treatment epidurally or along the spinal cord outside the thecal sac.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the various parts of the present invention, to include variations in size, materials, shape, form, function and manner of operation, assembly and use, are deemed readily apparent to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims of the present invention.

1. A system for providing pain relief to a person, comprising:
a spinal cord stimulation (SCS) generator adapted to provide an electrical stimulation signal;
an infusion pump adapted to pump fluid medication;
a controller for selectively controlling generation of said electrical stimulation from said SCS generator and for selectively controlling pumping of said medication with said infusion pump; and
a lead adapted for placement along the spinal cord of the person, said lead including:
(i) at least one electrode in electrical communication with said SCS generator for providing SCS to a first location on said spinal cord; and
(ii) an infusion port in fluid communication with said infusion pump for delivery of said medication to a second location on said spinal cord.

2. The system of claim 1 wherein said SCS generator, said infusion pump, and said controller are contained within a common housing.

3. The system of claim 1 wherein said SCS generator, said infusion pump, and said controller are adapted for implantation within the body cavity of the person.

4. The system of claim 1 wherein said SCS generator, said infusion pump, and said controller are adapted for remaining outside the body cavity of the person.

5. The system of claim 1 wherein said lead includes a plurality of electrodes.

6. The system of claim 5 wherein each of said plurality of electrodes is connected to said SCS generator using a wire.

7. The system of claim 1 wherein said infusion port is in fluid communication with said infusion pump using a lumen.

8. The system of claim 1 wherein said SCS generator and said infusion pump are contained within a common housing, wherein a plurality of electrodes are connected to said SCS generator using wires, wherein said infusion port is in fluid communication with said infusion pump using a lumen, and wherein said wires and said lumen are contained within a tubular element extending from said common housing to said lead.

9 The system of claim 1 wherein said first location is at approximately segment T10 of said spinal cord and said second location is at approximately segments L1 or L2 of said spinal cord.

10. The system of claim 1 wherein said first location is at approximately segment C3 or C4 of said spinal cord and said second location is at approximately segments C6 or C7 of said spinal cord.

11. The system of claim 1 wherein said second location is within the epidural space of said spinal cord.

12. The system of claim 1 wherein said second location is within the intrathecal sac of said spinal cord.

13. The system of claim 1 wherein said electrodes are distally located relative to said infusion port.

14. The system of claim 1 wherein said electrodes are proximally located relative to said infusion port.

15. The system of claim 1 wherein said lead further includes a steering lumen.

16. The system of claim 1 wherein said electrodes and said infusion port are within a paddle-shaped element adapted for placement along said spinal cord via open laminotomy or via a modified needle.

17. The system of claim 1 further comprising: (a) an electrode connector housing having at least one electrode connector lead and (b) an SCS adapter in electrical communication with said SCS generator, said SCS adapter having an electrode connector coupling adapted for engaging said at least one electrode when said electrode connector housing is engaged with said SCS adapter.

18. An apparatus adapted for placement along the spinal cord of a person for use in providing pain relief to the person, in combination with a spinal cord stimulation (SCS) generator adapted to provide an electrical stimulation signal and an infusion pump adapted to pump fluid medication, the SCS generator and the infusion pump each controlled by a controller for selectively controlling generation of the electrical stimulation from the SCS generator and for selectively controlling pumping of medication by the infusion pump, comprising:
a tubular housing;
a plurality of electrodes on an outer surface of said tubular housing, said electrodes in electrical communication with the SCS generator for providing SCS to a first location on the spinal cord; and
an infusion port extending through said tubular housing, said infusion port in fluid communication with the infusion pump for delivery of medication to a second location on said spinal cord.

19. The apparatus of claim 18 wherein each of said plurality of electrodes is connected to the SCS generator using a wire.

20. The apparatus of claim 18 wherein said infusion port is in fluid communication with the infusion pump using a lumen.

21. The apparatus of claim 18 wherein said electrodes are distally located relative to said infusion port.

22. The apparatus of claim 18 wherein said electrodes are proximally located relative to said infusion port.

23. The apparatus of claim 18 further comprising a steering lumen.

24. The apparatus of claim 18 wherein said electrodes and said infusion port are within a paddle-shaped element adapted for placement along the spinal cord via open laminotomy or via a modified needle.

25. A method of providing pain relief to a person, comprising the steps of:
implanting a combination infusion/spinal cord stimulation device along the spinal cord of the person;
infusing fluid medication at a first location of the spinal cord using the device; and
providing electrical stimulation at a second location of the spinal cord using the device.

26. The method of claim 25 wherein said steps of infusing fluid medication and providing electrical stimulation occur simultaneously.

27. The method of claim 25 wherein said steps of infusing fluid medication and providing electrical stimulation are repeated alternatively.

28. The method of claim 25 wherein said steps of infusing fluid medication and providing electrical stimulation occur serially.

29. The method of claim 25 wherein the first location is within the epidural space of the spinal cord.

30. The method of claim 25 wherein the first location is within the intrathecal sac of the spinal cord.
